(19) United States
(12) Patent Application Publication
(10) Pub. No.: US 2011/0046676 A1
(43) Pub. Date: Feb. 24, 2011

(54) DYNAMIC STABILIZATION ELEMENT FOR VERTEBRAE

(75) Inventors: Thomas Droulout, Chatou (FR); Dominique Petit, Verton (FR)

Correspondence Address: BACHMAN & LAPOINTE, P.C., 900 CHAPEL STREET, SUITE 1201, NEW HAVEN, CT, 06510, US

(73) Assignee: SPINEVISION, PARIS (FR)

(21) Appl. No.: 12/865,175

(22) PCT Filed: Feb. 4, 2009
(86) PCT No.: PCT/FR2009/000126

§ 371 Date: Nov. 16, 2010

(30) Foreign Application Priority Data
Feb. 4, 2008 (FR) 0850669

(51) Int. Cl. A61B 17/70

(52) U.S. Cl. 606/257; 606/264

(57) ABSTRACT

A dynamic stabilization member for the vertebrae, capable of interacting with at least two implantable connection assemblies is provided. Each connection assembly system including a system for anchoring into a vertebra and adapted for receiving the dynamic stabilization member, and a system for clamping the dynamic stabilization member (on the anchoring system), of the type that comprises a rod extending along a longitudinal axis and a cable provided with a casing made of an elastic material, characterized in that the cable comprises a fastening sheath surrounding the rod and including rigid areas spaced from each other.

### BACKGROUND

(1) Field of the Invention

The invention relates to the field of dynamic stabilization of vertebrae.

The invention more particularly relates to an element for the dynamic stabilization of neighbouring vertebrae, intended to cooperate with at least two spinal connexion sets implantable onto a vertebra.

(2) Prior Art

Generally speaking, dynamic stabilization elements are intended to realign vertebrae with respect to each other while reducing the constraints on the articular surfaces and the intervertebral discs, while enabling some motions of the vertebrae.

Such dynamic stabilization elements are known from the prior art.

More particularly, a dynamic stabilization element is known from the international patent application WO2004/024011, which is composed, at least partially, by a support made of a polymer material and two rods: a first rod which is substantially coaxial with the support and a second rod formed by turns surrounding the first rod, with said turns being at least partially buried in the support.

A flexible connexion element is also known from the international patent application WO2005/087121, which comprises a cable at least partially surrounded by a polymer envelope, with said cable being composed of at least an elastic strand coaxial with said envelope.

As they are intended to restore the alignment of vertebrae, the stabilization elements are fixed thereto through implantable connexion sets. Conventionally, connexion sets comprise bone anchoring means arranged to receive the dynamic stabilization element. The dynamic stabilization element is fixed onto the anchoring means through an additional closure part. Thus the dynamic stabilization element is held between the bone anchoring means and the closure part. The dynamic stabilization element is fixedly held on the anchoring means through the clamping of the dynamic stabilization element against the bone anchoring means. Clamping is generally provided by a nut which is placed in contact with the dynamic stabilization element. The dynamic stabilization element is pressed against the anchoring means under the clamping action of the nut.

A rigid protection ring is generally provided between the nut and the dynamic stabilization element, so as to provide the clamping of the nut on the dynamic stabilization element, and thus enable the holding thereof on the anchoring means. The presence of a protection ring between the nut and the dynamic connection element prevents a plastic deformation thereof thanks to the clamping operation.

Such configuration requires, however, adapting the length of the stabilization element and providing a precise position of the protection rings on the dynamic stabilization element, according to the position of the anchoring means. This may lead to a lengthy and tedious positioning of the dynamic stabilization elements.

### SUMMARY OF THE INVENTION

The invention more particularly aims at remedying the above mentioned drawback by providing a stabilization element which can be rapidly positioned onto the anchoring means while guaranteeing the requested elastic behaviour, or at least flexible behaviour, between the anchoring means.

For this purpose, and according to a first aspect, the invention relates to a dynamic stabilization element for vertebrae able to cooperate with at least two implantable connexion sets, each connection set comprising bone anchoring means, so arranged as to receive the dynamic stabilization element and means for clamping the dynamic stabilization element onto said anchoring means, the dynamic stabilization element comprising a rod extending along a longitudinal axis and comprising a cable provided with an envelope made of an elastic material. The dynamic stabilization element is remarkable in that it comprises a fixing sheath surrounding said rod, with said sheath comprising rigid zones spaced from each other.

The presence of a rigid fixing sheath makes it possible to provide and to maintain the clamping of the dynamic stabilization element on the anchoring means while enabling the extension, compression and flexion motions through the presence of spaces between the rigid zones of the fixing sheath.

The thus formed fixing sheath protects the flexible part of the stabilization element on every point over the length thereof, while preserving the flexion, distraction and/or compression properties of said element conferred by the very constitution of the rod.

Advantageously, the rigid zones are spaced from one another by a distance smaller than the nominal length of a contact zone defined by the means for clamping with the dynamic stabilization element.

The element provided with such a sheath further has the advantage of being rapidly positioned onto the anchoring means fixed on the vertebrae. As a matter of fact, the distance imposed between the rigid zones entails that the clamping means is mainly in contact with the rigid zones. The dynamic stabilization element thus requires no precise positioning on the anchoring means.

According to a particular configuration, the rigid zones consist of distinct rings spaced from one another by a distance smaller than the length of the contact zone.

According to another configuration, the fixing sheath consists of a helicoïdal strip comprising turns extending about the rod along an axis substantially coaxial with the longitudinal axis of said connection rod, said turns forming the rigid zones of the fixing sheath.

Advantageously, the dynamic stabilization element comprises means for holding the fixing sheath on the rod positioned between the rigid zones of the fixing sheath.

In order to damp a compression motion of the dynamic stabilization element, compression damping rings may be provided, with each ring being inserted between two adjacent rigid zones of the fixing sheath. According to a particular configuration, the damping rings form the fixing sheath holding means. In addition and advantageously, the damping means are composed of radial bulges of the envelope.

Thus, during the making of the dynamic stabilization element by moulding the elastic material about the cable, the elastic material intended to form the envelope is distributed into openings formed in the sheath, i.e. the spaces formed between the rigid zones. As it is “trapped” in the material composing the envelope, the fixing sheath is thus held firmly. The fixing sheath is thus prevented to slide thanks to the extension of the plastic material.

One or more lumens can also be provided in the rigid zones. The presence of lumens reinforces the holding functions of the parts “in excess” of the envelope (extensions). Such a configuration is particularly advantageous, especially when the fixing sheath is formed by distinct rings.

According to a particularly configuration of the dynamic stabilization element, the rigid zones are equidistant from each other.

In order to improve the global resistance, the free ends of the dynamic stabilization element are provided with rigid tips. According to a particular embodiment, the tips are preferably fixed by welding or stamping at the ends of the cables.

According to a second aspect, the invention relates to a connection element comprising at least one dynamic stabilization element as described above with the connection element being extended by at least a rigid rod. Depending on the desired application, it may be advantageous to provide a rigid rod running on from one end or both ends of the extended rod of the dynamic stabilization element. It will then be possible to provide both an osteosynthesis connection and a dynamic connection with only one connection element.

According to another aspect, the invention relates to a spinal fixing system comprising at least two implantable spinal connexion sets, with at least two connexion sets being connected by a dynamic stabilization element as described above.

### BRIEF DESCRIPTION OF THE DRAWINGS

Other objects and advantages of the invention will appear upon reading the following description referring to the appended drawings wherein:

FIG. 1 is a partial perspective view of a spinal fixing system comprising a dynamic stabilization element according to a first configuration of the invention held by two spinal connexion sets;

FIG. 2 is a schematic partial side view of the dynamic stabilization element according to the invention, which is in contact with means for clamping the connection sets;

FIG. 3 illustrates a partial cross-section of the dynamic stabilization element of FIG. 2, along axis III-III;

FIG. 4 illustrates a dynamic stabilization element according to a second configuration of the invention;

FIGS. 5a, 5b, 5c illustrate a hybrid connection element comprising at least a dynamic stabilization part;

FIGS. 6a and 6b respectively illustrate a perspective schematic view of a dynamic stabilization element according to a third configuration of the invention, with or without an envelope;

FIG. 7 illustrates a view of a dynamic stabilization element according to a fourth configuration of the invention, without an envelope;

FIGS. 8a and 8b respectively illustrate a perspective schematic view of a dynamic stabilization element according to a fifth configuration of the invention with and without an envelope; and

FIG. 9 illustrates a view of a dynamic stabilization element according to a sixth configuration of the invention which is shown without an envelope.

### DETAILED DESCRIPTION OF THE PREFERRED EMBODIMENT(S)

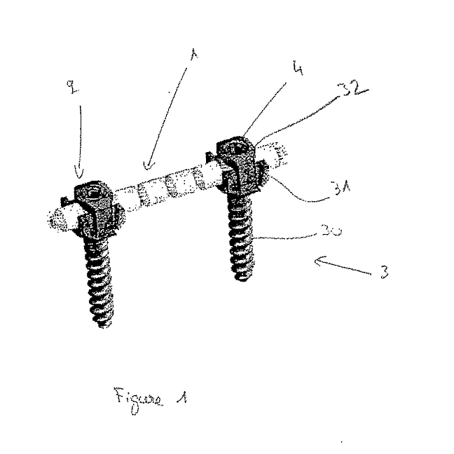

In reference with FIGS. 1 to 3, a neighbouring vertebrae dynamic stabilization element is described. The dynamic stabilization element 1 is intended to be held along the vertebrae using at least two implantable spinal connexion sets 2.

Conventionally, a connexion set 2 comprises bone anchoring means 3 so arranged as to receive the dynamic stabilization element 1 and means for clamping 4 the dynamic stabilization element 1 onto said anchoring means 2.

In the described embodiment, the anchoring means 3 comprises a threaded part 30 intended for the anchoring into the vertebra, having a U-shaped head 31 on the top thereof, intended for receiving the dynamic stabilization element 1, the bottom of the U defining a zone for receiving the dynamic stabilization element 1. The dynamic stabilization element 1 is held in position at the bottom of the U of the head 31 using a closure part 32. The head 31 of the anchoring means 3 and the closure part 32 are configured for mutually cooperating by snapping.

The means for clamping 4 the dynamic stabilization element 1 into the head 3 consists of an element forming a nut or a clamping screw intended to be accommodated in a through-hole arranged in the closure part 32. When accommodated in the cavity of the closure part 32, the clamping means 4 rests against the dynamic stabilization element 1 and clamps said element against the bottom of the U of the head 31. Advantageously the cavity has a shape matching that of the clamping means 4.

It should, of course, be understood that the configuration of the anchoring means is given as an example and that the invention is not limited to such a configuration. More particularly, the head 31 may be provided as a part separate from the anchoring means 3, of a conventional type in the spinal connection systems.

The dynamic stabilization element 1 is in the form of a rod 5 extending along a longitudinal axis A, and the rod comprises a cable 6 surrounded by an elastic material envelope 7. Such a constitution thus makes it possible to confer the required flexibility to enable a dynamic connexion of vertebrae together. Advantageously, the cable is made of titanium and the envelope 7 is made of polymer, such as urethane polycarbonate.

The connexion element 1 further comprises a fixing sheath 8 comprising rigid zones 9 positioned successively. Such rigid zones 9 are spaced from each other at a distance which is sufficient for enabling the flexion of said rod 5. The flexible behaviour of the rod 5 is thus preserved.

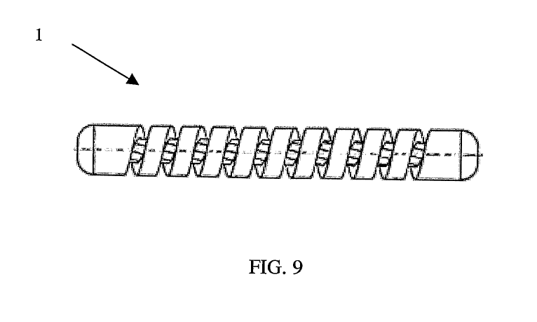

In the described embodiment, the fixing sheath 8 consists of independent and distinct rings 10 which are fixed on the envelope 7. Of course this is a particular exemplary embodiment since the fixing sheath 8 may have any other arrangement enabling the formation of spaced rigid zones, such as a sheath having a helicoïdal shape (FIG. 9).

As previously seen, the rod 5 is positioned at the bottom of the U of the head 31 of the anchoring means 3, and held “fixed” therein by the clamping means 4 which comes to rest in contact with the rod 5. The clamping means 4 defines a contact area 11 with the rod 5. The contact zone 11 is characterized by its nominal length.

The distance between said rings 10 is determined so as to be smaller than the nominal length of the contact zone 11, so that the clamping force of the clamping means 4 can mainly be exerted on the rings 10. Thus the rod 5 needs no specific positioning on the connexion sets, since the means for clamping each connexion set mainly exerts a pressure on the rings 10, whatever the positions thereof on the rod 5.

FIG. 2 illustrates an exemplary configuration of such a rod in which, in order to facilitate the understanding, the means for clamping 4 three connexion sets only are shown. In this example, the rod 5 comprises rings 10 having a length of 5 millimetres. Such rings 10 are positioned on the envelope 7 of the rod 5, at a regular distance from each other. Each ring 10 is spaced from the adjacent rings by a distance of 2 millimetres. The clamping means 4 shown have a substantially circular shape. The contact face of the clamping means 4 with the connexion element 1 advantageously has a diameter of 5 millimetres. The clamping means 4 rests on the rod 5 and forms contact zones 11 having a nominal length of 5 millimetres, i.e. a length which is greater than the spacing between each ring 10. Thus the pressure applied by the clamping means 4 onto the rod 5 is exerted on the rings 10, whatever the position of the clamping means 4 on the rod 5.

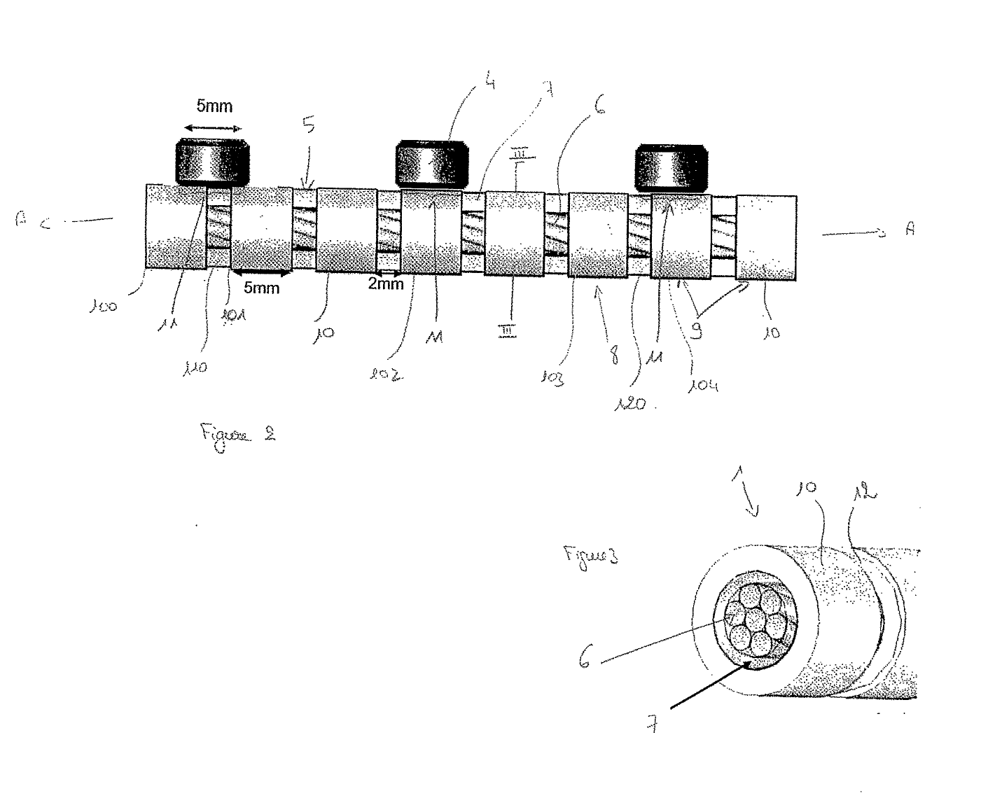

The rings 10, and, more widely the fixing sheath 8 are blocked on the envelope 7. As a matter of fact, during the manufacturing of the dynamic stabilization element 1, the material the envelope 7 is made of runs into the spaces formed between the cable and the rings 10. The rings 10 are then held distant from each other and blocked by the radial bulges 12 of the envelope 7 formed between said rings (FIG. 3).

The presence of bulges 12 made of plastic material offers two advantages. On the one hand, the bulges 12 make it possible to trap the rings, as just seen, thus preventing the sliding thereof on the envelope 7. On the other hand, as the bulges 12 are positioned between the rigid zones of the fixing sheath, they respectively make zones damping the extension, compression and flexion motions of the dynamic stabilization element 1.

According to an advantageous configuration, the rings 10 comprise lumens 14 (FIG. 4). The presence of such lumens improves the holding of the rings 10 on the rod 5. They reinforce the holding function of the bulges 12 of the envelope 7.

FIG. 2 illustrates all the possible positions of the clamping means 4 on the rod 5. The first clamping means (located far left on the rod) tops two adjacent rings 100, 101 on the rod 5. The first clamping means thus has a contact surface covering portions of two neighbouring rings 100, 101 and the space 110 arranged between the two rings 100, 101. The clamping force is exerted on the rings 100, 101 thanks to the rigidity thereof. The second clamping means (central clamping means) is totally in contact with a ring of the rod 5 (ring 102). The clamping force exerted by the central clamping means is thus applied onto the only one ring concerned. The third clamping means (located far right on the rod) partially tops the space 120 arranged between the rings 103, 104 and said ring 104. Then again, the clamping force is exerted on the ring 104 only thanks to the rigidity thereof.

The clamping means 4 so arranged provide sufficient clamping and holding of the rod 5 on the anchoring means 3.

In addition, and advantageously, the free ends 15, 16 of the dynamic stabilization element 1 are respectively provided with rigid tips 17, 18. According to a particular embodiment, the tips 17, 18 are fixed preferably by welding or stamping to said ends.

According to a particular configuration, one or more ring(s) is/are mounted to slide on the rod 5.

FIGS. 1 to 4 illustrate a dynamic stabilization element comprising a fixing sheath 7 the rigid zones 9 of which have the same length and are equidistant from each other. It should obviously be understood that the invention is not limited to such a configuration and that rigid zones may be provided which have different dimensions and/or rigid zones whose spacing can be different from one ring to another (not shown), it being understood that the distance between each ring must remain smaller than the nominal length of the contact zone.

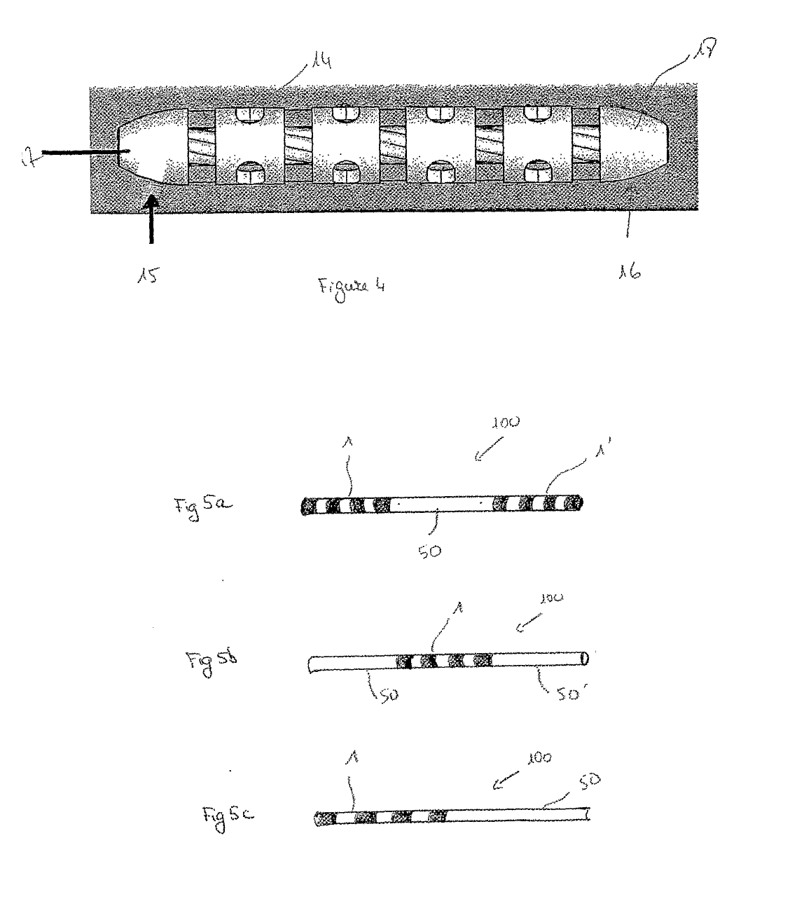

In addition, at least one of the ends dynamic stabilization element 1 may be extended by a rigid element (FIG. 5b and FIG. 5c), which may also be extended by another dynamic stabilization element 1 (FIG. 5a), so as to form a hybrid connection element 100 providing both an osteosynthesis connection and a dynamic connection. More particularly, the connection element 100 of FIG. 5a is provided with two parts 1, 1′ providing a dynamic connection and connected by a part providing an osteosynthesis connection 50. The connection element 100 of FIG. 5b illustrates a connection element provided with two parts 50, 50′ providing an osteosynthesis connection and connected by a part providing a dynamic connection 1. It should obviously be understood that the invention is not limited to such an arrangement of elements, and that a connection element composed of a succession of osteosynthesis connection elements and dynamic connection elements may be provided for.

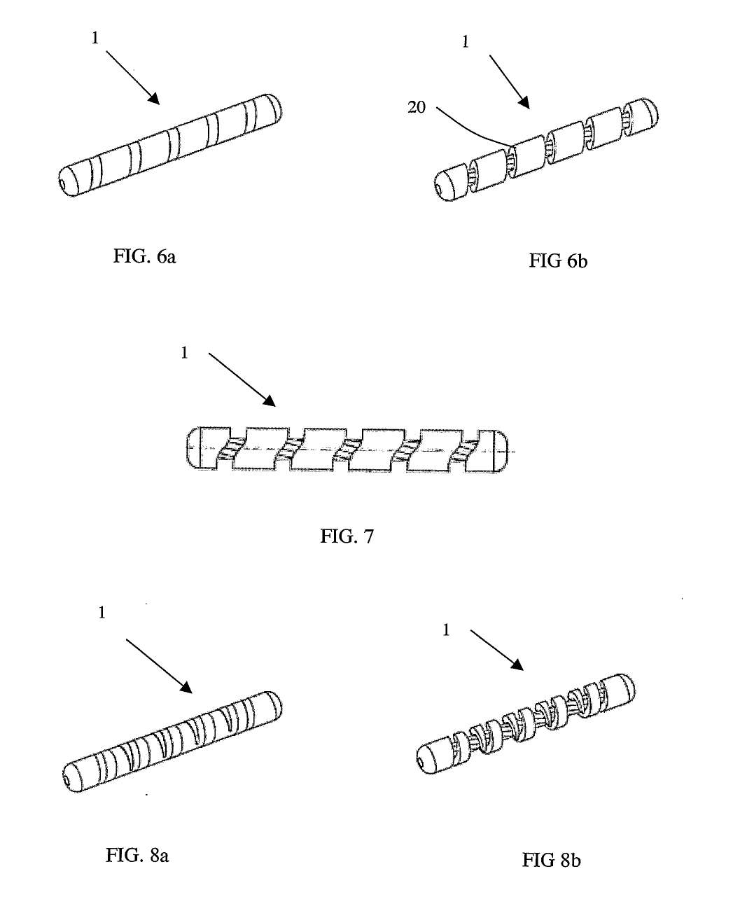

In the embodiment described here above, the fixing sheath is formed by rings 10, with each one of the rings 10 having end faces 20 perpendicular to the longitudinal axis 1 of the rod 5. In order to improve the torsion resistance of the dynamic connection element 1, it is advantageous to provide rings 10 configured to have, respectively, end faces 20 inclined with respect to the longitudinal axis A of the rod 5, with the end faces 20 of each ring 10 being positioned parallel with respect to each other (FIGS. 6a and 6b).

In FIG. 6b, the end faces 20 are shown as plane. It should obviously be understood that this is a particular exemplary embodiment, since the ends of each ring 10 may have any surface, as illustrated in FIG. 7, as an example.

Rings 10 having a helicoïdal shape (FIGS. 8a and 8b) can also be provided. Such a configuration of the fixing sheath 8 makes it possible to improve the compression and extension properties of the dynamic connection element 1. Advantageously, the helicoïdal pitch and the number of turns of the so configured rings 10 are defined according to the desired behaviour of the dynamic connection element 1 in response to a compression or extension solicitation.

The invention is described above as an example. It should be understood that the persons skilled in the art may bring various modifications to the invention without leaving the scope thereof.

1-10. (canceled)

11. A dynamic stabilization element for vertebrae which cooperates with at least two implantable connexion sets, each connection set comprising means for anchoring into a vertebra and being arranged to receive the dynamic stabilization element and means for clamping the dynamic stabilization element onto said anchoring means, the dynamic stabilization element comprising a rod extending along a longitudinal axis, a cable provided with an envelope made of an elastic material a fixing sheath comprising rigid zones spaced from each other surrounding the rod, and rings for damping a motion of the stabilization element, and the rings being composed of radial bulges of the envelope respectively placed between rigid zones adjacent to the fixing sheath.

12. A dynamic stabilization element according to claim 1, wherein the rigid zones are spaced from one another by a distance smaller than a nominal length of a contact zone defined by the means for clamping with the dynamic stabilization element.

13. A dynamic stabilization element according to claim 1, wherein the rigid zones consist of distinct rings.

14. A dynamic stabilization element according to claim 1, wherein the fixing sheath consists of an helicoïdal strip comprising turns extending about the rod along an axis substantially coaxial with the longitudinal axis of said rod, and said turns forming the rigid zones of the fixing sheath.

15. A dynamic stabilization element according to claim 1, wherein the rigid zones comprise at least one lumen for facilitating the fixing of the fixing sheath on the envelope.

16. A dynamic stabilization element according to claim 1, wherein the rigid zones are equidistant from each other.

17. A dynamic stabilization element according to claim 1, wherein the stabilization element comprises means for holding the fixing sheath on the rod, with the fixing sheath holding means being formed by the damping rings.

18. A dynamic stabilization element according to claim 1, further comprising rigid tips fixed at each end of the cable.

19. A connection element comprising at least one dynamic stabilization element according to claim 1, with the dynamic stabilization element extending with at least one rigid rod.

20. A spinal fixing system comprising at least two implantable spinal connection sets, with both connection sets at least being connected by a dynamic stabilization element according to claim 1.
